(19) United States
(12) Patent Application Publication
(10) Pub. No.: US 2023/0174668 A1
(43) Pub. Date: Jun. 8, 2023

(54) COMPOSITIONS AND METHODS FOR TREATING LYSOSOMAL STORAGE DISORDERS

(71) Applicants: THE UNITED STATES GOVERNMENT AS REPRESENTED BY THE DEPARTMENT OF VETERANS AFFAIRS, Washington, DC (US); THE BOARD OF TRUSTEES OF THE LELAND STANFORD JUNIOR UNIVERSITY, Stanford, CA (US)

(72) Inventors: John Vincent PLUVINAGE, Atherton, CA (US); Anton WYSS-CORAY, Palo Alto, CA (US)

(21) Appl. No.: 17/924,612

(22) PCT Filed: May 18, 2021
(86) PCT No.: PCT/US2021/032875

§ 371 Date: Nov. 10, 2022

Related U.S. Application Data
(60) Provisional application No. 63/026,448, filed on May 18, 2020.

(51) Int. Cl. C07K 16/28; A61P 3/00

(52) U.S. Cl. CPC C07K 16/2896; C07K 16/2863; A61P 3/00

(57) ABSTRACT

Provided herein are compositions and methods to treat lysosomal storage disorders. In particular, provided herein are compositions, methods, kits and uses for inhibition or modification of soluble CD22 (sCD22) and its ligand interactions as therapeutic targets in lysosomal storage disorders including Niemann-Pick Disease Type C.

### CROSS-REFERENCE TO RELATED APPLICATIONS

This application claims the benefit of U.S. Provisional Application No. 63/026,448, filed May 18, 2020, the contents of which are fully incorporated herein by reference.

### STATEMENT REGARDING FEDERALLY SPONSORED RESEARCH

This invention was supported by Grant Nos. IO1BX004544 awarded by the Veteran's Administration, and NIH RF1AG064897 awarded by the National Institutes of Health. The U.S. government has certain rights in the invention.

### FIELD

Provided herein are compositions and methods to treat lysosomal storage disorders. In particular, provided herein are compositions, methods, kits and uses for inhibition or modification of soluble CD22 (sCD22) and its ligand interactions as therapeutic targets in lysosomal storage disorders including Niemann-Pick Disease Type C.

### BACKGROUND

Lysosomes are specialized organelles¹ that degrade and recycle macromolecules, connecting catabolic and anabolic metabolism. While all tissues require lysosomes to maintain homeostasis, the metabolically demanding brain is especially reliant on lysosome function as evident by the neurological phenotypes of many lysosomal storage diseases (LSDs). Neuropathological changes in rare inherited LSDs resemble those in common age-related neurodegenerative diseases. Both entities may reflect accumulation of lysosomal cargo due to increased substrate production, impaired substrate degradation, or reduced clearance of end products. For example, autosomal recessive mutations in beta-glucocerebrosidase (GBA), that encodes a lysosomal enzyme necessary for glycosphingolipid degradation, cause Gaucher's disease, whereas heterozygous carriers are predisposed to Parkinson's disease². Even when the underlying genetic etiology is different, LSDs and age-related neurodegenerative diseases display molecular overlap. For example, Niemann Pick Disease Type C (NPC), an LSD characterized by lysosomal accumulation of unesterified cholesterol, and Alzheimer's disease (AD) share similarities including protein aggregation, neuroinflammation, and compensatory upregulation of lysosomal genes^{3,4}.

NPC is caused by mutations in either NPC1 or NPC2 which impair cholesterol efflux from the lysosome. Patients typically present in early childhood with motor deficits and gradual cognitive impairment. These clinical signs are associated with neurodegeneration, especially in the cerebellum, and demyelination⁵. Progressive neurological decline leads to premature death in the second or third decade⁶. As in AD, investigational therapies for NPC have shown early promise in preclinical animal models, but have failed in human trials.

Among cell types affected in NPC and AD, microglia (brain-resident macrophages) are particularly vulnerable to lysosome dysfunction due to the phagocytic stress of clearing dying neurons and debris from the degenerating brain. Consequently, microglia upregulate proinflammatory gene expression modules that drive neuroinflammation. Although NPC1/2 loss-of-function in neurons may initiate neurodegeneration in NPC, genetic ablation of interferon regulatory factor 8 (Irf8), a transcription factor necessary for microglial activation, reduces Purkinje cell death, ameliorates cognitive decline, and extends lifespan in a mouse model of NPC⁷. However, recent single cell and single nucleus RNAseq (snRNA-seq) atlases have revealed low molecular overlap between mouse and human microglia^{8,9,10}, therefore differences between mice and humans must be balanced prior to translational development of microglia-directed therapies.

Accordingly, compositions and methods for restoring lysosomal dysfunction in human LSDs including NPC mediated by sCDD are needed.

### SUMMARY

Provided herein are compositions and methods to treat lysosomal storage disorders. In particular, provided herein are compositions, methods, kits and uses for inhibition or modification of soluble CD22 (sCD22) and its ligand interactions as therapeutic targets in lysosomal storage disorders including Niemann-Pick Disease Type C.

Cluster of differentiation-22 (CD22), a molecule belonging to the Siglec family of lectins, is a sugar binding transmembrane protein that specifically binds sialic acid, with an immunoglobulin (Ig) domain located at its N-terminus. Presence of Ig domains establish CD22 as a member of the immunoglobulin superfamily. CD22 is upregulated on microglia in the aging mouse brain where it inhibits phagocytosis of protein aggregates and myelin debris¹¹. CD22 blockade restores microglial homeostasis and improves cognitive function in aged mice. Soluble CD22 (sCD22) is increased in the cerebrospinal fluid (CSF) of NPC patients⁷.

Provided herein are methods of treating a lysosomal storage disorder, comprising: exposing a subject's microglia to a soluble CD22 (sCD22) inhibitor wherein the exposing treats the lysosomal storage disorder. In other embodiments, provided herein are methods of treating a lysosomal storage disorder comprising: exposing a subject's neurons, sensory neurons, receptor neurons, motor neurons, interneurons, principal neurons, local circuit neurons, macroglia, astrocytes, oligodendrocytes, or other central nervous system cell to a soluble sCD22 inhibitor wherein the exposing treats the lysosomal storage disorder. In some embodiments, the subject is a human subject. In other embodiments, the lysosomal storage disorder is Niemann-Pick Disease Type C (NPC). In certain embodiments, the sCD22 inhibitor is an anti-sCD22 antibody. In particular embodiments, the sCD22 inhibitor is a sCD22 antagonist. In further embodiments, the sCD22 antagonist is a glycomimetic compound. In still further embodiments, the sCD22 antagonist is selected from the group consisting of a small molecule, a peptide, and a nucleic acid. In given embodiments, the small molecule inhibitor is a multivalent conjugate. In specific embodiments, the multivalent conjugate comprises a glycan component and/or a glycomimetic component. In some embodiments, the multivalent conjugate is a neoglycoprotein, a glycopolymer, a glycol-liposome, or a nanoparticle. In additional embodiments, the sCD22 inhibitor interferes with sCD22 expression. In other embodiments, the exposing is in vivo exposing, ex vivo exposing or in vitro exposing. In certain embodiments, the exposing to the sCD22 inhibitor is selected from the group consisting of local administration, topical administration, intrathecal administration, intraparenchymal administration, intracerebroventrical administration, intravenous administration, intraarterial administration, intrapulmonary administration and oral administration. In further embodiments, the exposing comprises combination therapy with an agent that interferes with NPC1 and/or NPC2 expression.

In some embodiments, provided herein are methods of treating a lysosomal storage disease in a subject, comprising; a) assaying a cerebrospinal fluid (SCF) sample from a subject; and b) administering an agent that decreases sCD22.

In some embodiments, provided herein are methods of treating a lysosomal storage disorder, comprising: exposing a subject's microglia to an insulin-like growth factor 2 receptor (IGF2R) inhibitor wherein the exposing treats the lysosomal storage disorder. In other embodiments, provided herein are methods of treating a lysosomal storage disorder comprising: exposing a subject's neurons, sensory neurons, receptor neurons, motor neurons, interneurons, principal neurons, local circuit neurons, macroglia, astrocytes, oligodendrocytes, or other central nervous system cell to an insulin-like growth factor 2 receptor (IGF2R) inhibitor wherein the exposing treats the lysosomal storage disorder. In some embodiments, the subject is a human subject. In other embodiments, the lysosomal storage disorder is Niemann-Pick Disease Type C (NPC). In particular embodiments, the IGF2R inhibitor is an IGF2R antagonist. In other embodiments, the IGF2R inhibitor blocks the ability of sCD22 to bind to IGF2R, but otherwise spares IGF2R function in the same cell or in other cells. For example, in such embodiments, the inhibitor is selective for preventing the interaction of CD22 to IGF2R, but does not deleteriously interfere with IGR2R activity in the absence of CD22. In further embodiments, the IGF2R inhibitor is an anti-IGF2R antibody. In particular embodiments, the anti-IGF2R antibody is a recombinant Anti-M6PR (cation independent) antibody [EPR6599] (ab124767) (Abcam). In still further embodiments, the IGF2R inhibitor is an IGF2R antagonist. In another embodiment, the IGF2R antagonist is a glycomimetic compound. In specific embodiments, the IGF2R antagonist is selected from the group consisting of a small molecule, a peptide, and a nucleic acid. In certain embodiments, the IGF2R inhibitor interferes with IGF2R expression. In additional embodiments, the exposing to the IGF2R inhibitor is selected from the group consisting of local administration, topical administration, intrathecal administration, intraparenchymal administration, intracerebroventrical administration, intravenous administration, intraarterial administration, intrapulmonary administration and oral administration. In some embodiments, the exposing comprises combination therapy with an agent that interferes with NPC1 and/or NPC2 expression.

Provided herein are compositions, comprising: a) a sCD22 antagonist; and b) an IGF2R antagonist. In some embodiments, the compositions are administered to a subject. In some embodiments, the composition is a kit comprising each antagonist. In some embodiments, the kit comprises a container having both antagonists formulated together for simultaneous administration to a subject. In some embodiments, the kit comprises multiple containers each separately containing a distinct antagonist whereby the antagonists can be administered to a subject individually, or together, either simultaneously or in sequence.

In some embodiments, provided herein are methods of treating a central nervous system disorder, comprising: exposing a subject's one or more central nervous system cell types to a sCD22 antagonist wherein said exposing treats the central nervous system disorder. In other embodiments, the one or more central nervous system cell type is a neuron, a sensory neuron, a receptor neuron, a motor neuron, an interneuron, a principal neuron, a local circuit neuron, a microglia, a macroglia, an astrocyte, an oligodendrocyte, or other central nervous system cell. In particular embodiments, the one or more central nervous system cell type is an oligodendrocyte. In certain embodiments, the central nervous system disorder is a lysosomal storage disorder, for example, Niemann-Pick disease, Niemann-Pick disease Type C, Fabry disease, Gaucher disease, Tay-Sachs disease, Krabbe disease, metachromatic leukodystrophy, Hurler syndrome, and Hunter syndrome. In further embodiments, the central nervous system disorder is a neurodegenerative disorder. In still further embodiments, the neurodegenerative disorder is selected from the group consisting of, for example, dementia, frontotemporal dementia, Parkinson's disease, Alzheimer's disease, amyotrophic lateral sclerosis, Huntington's disease, Nasu-Hakola disease, stroke, systemic lupus erythematosus, central nervous system (CNS) lupus, granulomatous disease, sarcoidosis, spinal cord injury, traumatic brain injury, multiple system atrophy, Lewy body dementia, lymphocytic choriomeningitis virus infection, viral and bacterial infections of the CNS, Smith-Lemli-Opitz syndrome, Tay-Sachs, schizophrenia, major depressive disorder, bipolar disorder, anorexia nervosa, obsessive compulsive disorder, general anxiety disorder, and/or other disorder with lysosomal dysfunction. In certain embodiments, the sCD22 inhibits IGF2R. While many of the exemplary embodiments described herein make reference to sCD22, it should be understood that the invention is not limited to sCD22.

In some embodiments, provided herein are methods of treating a central nervous system disorder, comprising: exposing a subject's one or more central nervous system cell types to a membrane bound CD22 antagonist wherein said exposing treats the central nervous system disorder. In other embodiments, the one or more central nervous system cell type is a neuron, a sensory neuron, a receptor neuron, a motor neuron, an interneuron, a principal neuron, a local circuit neuron, a microglia, a macroglia, an astrocyte, an oligodendrocyte, or other central nervous system cell. In particular embodiments, the one or more central nervous system cell type is an oligodendrocyte. In other embodiments, the central nervous system disorder is a lysosomal storage disorder, for example, Niemann-Pick disease, Niemann-Pick disease Type C, Fabry disease, Gaucher disease, Tay-Sachs disease, Krabbe disease, metachromatic leukodystrophy, Hurler syndrome, and Hunter syndrome. In further embodiments, the central nervous system disorder is a neurodegenerative disorder. In still further embodiments, the neurodegenerative disorder is selected from the group consisting of, for example, dementia, frontotemporal dementia, Parkinson's disease, Alzheimer's disease, amyotrophic lateral sclerosis, Huntington's disease, Nasu-Hakola disease, stroke, systemic lupus erythematosus, central nervous system (CNS) lupus, granulomatous disease, sarcoidosis, spinal cord injury, traumatic brain injury, multiple system atrophy, Lewy body dementia, lymphocytic choriomeningitis virus infection, viral and bacterial infections of the CNS, Smith-Lemli-Opitz syndrome, Tay-Sachs, schizophrenia, major depressive disorder, bipolar disorder, anorexia nervosa, obsessive compulsive disorder, general anxiety disorder, and/or other disorder with lysosomal storage dysfunction. In certain embodiments, the membrane bound CD22 inhibits IGF2R. While many of the exemplary embodiments described herein make reference to membrane bound CD22, it should be understood that the invention is not limited to membrane bound CD22.

In some embodiments, provided herein are methods of treating a central nervous system disorder, comprising: exposing a subject's one or more central nervous system cell types to a ST6GAL1 inhibitor wherein said exposing treats the central nervous system disorder. ST6GAL1 inhibitors are described, for example, at Manhart et al. J Biol Chem 292(33): 2017; 13514-13520, and Lee et al. J Biol Chem 289(13); 2014; 8742-8748, incorporated by reference herein in their entirety.

### DESCRIPTION OF THE FIGURES

FIG. 1 shows that oligodendrocyte-derived sCD22 binds to ligands on microglia. (A) t-SNE dimential reduction of snRNA-seq data from 48 human donors colored by cell-type annotations (left) and CD22 expression (right) (from Allen Brain Atlas). (B) Representative images of age-matched control (top) and NPC cerebella (bottom) probed for proteolipid protein 1 PLP1 (red) and CD22 (blue). Quantification shows the percentage of PLP1+ oligodendrocytes that co-express CD22. (C) UCSC genome browser view showing location of RNA-seq counts within CD22 locus from blood and unique brain regions (from GTEX project). (D) RT-PCR followed by PAGE analysis showing predicted and truncated (TM-null) bands amplified from cortex and cerebellum. (E) t-SNE dimential reduction of snRNA-seq data from 48 human donors colored by ST6 beta-galactoside alpha-2,6-sialyltransferase 1 ST6GAL1 expression (from Allen Brain Atlas). (F) Representative flow cytometry analysis of microglia isolated from fresh human autopsy specimen pre-treated with or without sialidase and stained with isotype control antibody or fluorescent recombinant CD22. (G) Schematic of differences between human and mouse CD22 in the brain.

FIG. 2 shows that genetic and proteomic screens identify IGF2R as a CD22 ligand. (A) CRISPR-Cas9 screening strategy for sCD22 ligands on U937 cells. (B) Volcano plot showing significant hits that inhibit (blue) or promote (red) sCD22 binding. (C) Flow cytometry validation of IGF2R hit showing ablation of sCD22 binding to IGF2R KO cells. (D) AP-MS screening strategy for direct binding partners of sCD22 on the surface of U937 cells. (E) Volcano plot showing proteins enriched (red) and depleted (blue) in sCD22 bound fraction.

FIG. 3 shows that CD22 blocks IGF2R at M6P binding sites and inhibits lysosomal trafficking. (A) Schematic of assay for exogenous M6P-dependent lysosomal protein trafficking. (B) Trafficking of NPC2 protein from extracellular space to lysosome in U937s pretreated with M6P (grey), sCD22-delta (black), sCD22 (red), and sCD22+anti-IGF2R (blue), assessed over 24 hours. (C) Trafficking of CTSD protein from extracellular space to lysosome in U937s pretreated with M6P (grey), sCD22-delta (black), sCD22 (red), and sCD22+anti-IGF2R (blue), assessed over 24 hours.

FIG. 4 shows that CD22 antibodies disrupt sCD22-IGFIIR interactions. (A) CD22-blocking mAb generation strategy. (B) Supernatants from 51 hybridoma clones were preincubated with sCD22, then exposed to U937 cells expressing IGF2R. Highlighted clones demonstrate potent blocking activity.

### DEFINITIONS

To facilitate an understanding of the present disclosure, a number of terms and phrases are defined below:

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “non-human animals” refers to all non-human animals including, but not limited to, vertebrates such as rodents, non-human primates, ovines, bovines, ruminants, lagomorphs, porcines, caprines, equines, canines, felines, ayes, etc.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., Alzheimer's disease, Parkinson's disease, atherosclerosis, cancer). Test compounds comprise both known and potential therapeutic compounds. A test compound can be determined to be therapeutic by screening using the screening methods of the present disclosure.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, and industrial samples. Such examples are not however to be construed as limiting the sample types applicable to the present disclosure.

As used herein, the term “effective amount” refers to the amount of a compound (e.g., a compound described herein) sufficient to effect beneficial or desired results. An effective amount can be administered in one or more administrations, applications or dosages and is not limited to or intended to be limited to a particular formulation or administration route.

As used herein, the term “co-administration” refers to the administration of at least two agent(s) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

As used herein, the term “pharmaceutical composition” refers to the combination of an active agent with a carrier, inert or active, making the composition especially suitable for diagnostic or therapeutic use in vivo, or ex vivo.

As used herein, the term “antigen binding agent” (e.g., “antigen-binding protein” or protein mimetic such as an apatamer) refers to proteins that bind to a specific antigen. “Antigen-binding proteins” include, but are not limited to, immunoglobulins, including polyclonal, monoclonal, chimeric, single chain, single domain, scFv, minibody, nanobody, and humanized antibodies, Fab fragments, F(ab′)2 fragments, and Fab expression libraries.

As used herein, the term “single-chain variable fragment” (scFv) refers to an antibody fragment that comprises a fusion protein of the variable regions of the heavy (V_{H}) and light chains (V_{L}) of an immunoglobulin. In some embodiments, the V_{H} and V_{L} are connected with a short linker peptide.

As used herein, the term “minibody” refers to an antibody fragment that retains antigen binding activity. In some embodiments, minobodies comprise an scFv fused to an Fc region (e.g., an IgG Fc region).

Various procedures known in the art are used for the production of polyclonal antibodies. For the production of antibody, various host animals can be immunized by injection with the peptide or protein containing the desired epitope including but not limited to rabbits, mice, rats, sheep, goats, llamas, alpacas, etc. In a preferred embodiment, the peptide is conjugated to an immunogenic carrier (e.g., diphtheria toxoid, bovine serum albumin (BSA), or keyhole limpet hemocyanin (KLH)). Various adjuvants are used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, Gerbu adjuvant and potentially useful human adjuvants such as BCG (Bacille Calmette-Guerin) and Corynebacterium parvum.

For preparation of monoclonal antibodies, any technique that provides for the production of antibody molecules by continuous cell lines in culture may be used (See e.g., Harlow and Lane, Antibodies: A Laboratory Manual, Cold Spring Harbor Laboratory Press, Cold Spring Harbor, N.Y.). These include, but are not limited to, the hybridoma technique originally developed by Köhler and Milstein (Köhler and Milstein, Nature, 256:495-497 [1975]), as well as the trioma technique, the human B-cell hybridoma technique (See e.g., Kozbor et al., Immunol. Today, 4:72 [1983]), and the EBV-hybridoma technique to produce human monoclonal antibodies (Cole et al., in Monoclonal Antibodies and Cancer Therapy, Alan R. Liss, Inc., pp. 77-96 [1985]). In other embodiments, suitable monoclonal antibodies, including recombinant chimeric monoclonal antibodies and chimeric monoclonal antibody fusion proteins are prepared as described herein.

According to the invention, techniques described for the production of single chain antibodies (U.S. Pat. No. 4,946,778; herein incorporated by reference) can be adapted to produce specific single chain antibodies as desired. An additional embodiment of the invention utilizes the techniques known in the art for the construction of Fab expression libraries (e.g., Huse et al., Science, 246:1275-1281 [1989]) to allow rapid and easy identification of monoclonal Fab fragments with the desired specificity.

In some embodiments, monoclonal antibodies are generated using the ABL-MYC method (See e.g., U.S. Pat. Nos. 5,705,150 and 5,244,656, each of which is herein incorporated by reference) (Neoclone, Madison, Wis.). ABL-MYC is a recombinant retrovirus that constitutively expresses v-abl and c-myc oncogenes. When used to infect antigen-activated splenocytes, this retroviral system rapidly induces antigen-specific plasmacytomas. ABL-MYC targets antigen-stimulated (Ag-stimulated) B-cells for transformation.

In some embodiments, biopanning as described in Pardon et al., Nat Protoc. 2014 March; 9(3):674-93 is used to generate single domain antibodies. In some embodiments, to generate murine scFv units, phage-based biopanning strategies, of which there are several published protocols available, are used.

Antibody fragments that contain the idiotype (antigen binding region) of the antibody molecule can be generated by known techniques. For example, such fragments include but are not limited to: the F(ab′)2 fragment that can be produced by pepsin digestion of an antibody molecule; the Fab′ fragments that can be generated by reducing the disulfide bridges of an F(ab′)2 fragment, and the Fab fragments that can be generated by treating an antibody molecule with papain and a reducing agent.

Genes encoding antigen-binding proteins can be isolated by methods known in the art. In the production of antibodies, screening for the desired antibody can be accomplished by techniques known in the art (e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), Western Blots, precipitation reactions, agglutination assays (e.g., gel agglutination assays, hemagglutination assays, etc.), complement fixation assays, immunofluorescence assays, protein A assays, phage display biopanning, and immunoelectrophoresis assays, etc.)

As used herein, the term “toxic” refers to any detrimental or harmful effects on a cell or tissue as compared to the same cell or tissue prior to the administration of the toxicant.

### DETAILED DESCRIPTION OF THE DISCLOSURE

Provided herein are compositions and methods to treat lysosomal storage disorders. In particular, provided herein are compositions, methods, kits and uses for inhibition or modification of soluble CD22 (sCD22) and its ligand interactions as therapeutic targets in lysosomal storage disorders including Niemann-Pick Disease Type C.

Experiments conducted in the course of development of certain embodiments of the present invention denote the source, target, and function of sCD22 in the human brain. Specifically, data herein establishes that sCD22 is a cerebellum-specific isoform secreted by oligodendrocytes that binds IGF2R on microglia, and that impairs lysosomal function. Further, specific CD22-blocking mAbs are shown to rescue lysosome dysfunction in NPC cells. While CD22 is highly conserved at the sequence level, a soluble isoform is identified herein that is expressed in oligodendrocytes in humans, compared to a membrane-bound form in microglia in mice. These differences reinforce the utility of humanized models for target LSD and NPC discovery and drug development. Accordingly, targeting sCD22 in the central nervous system (CNS) provides a therapeutic approach for human NPC comprising targeting sCD22 directly with, for example, intrathecal mAbs and additional anti-sCD22 compounds to slow disease progression in NPC. Because sCD22 impairs exogenous lysosomal protein trafficking via IGF2R, CD22 blockade further serves as an adjuvant for enzyme replacement therapy for other LSDs. While the description below focuses on antibodies, it should be understood that any effective antagonist of sCD22 may be used.

### Antibodies

In some embodiments, the present disclosure provides antibodies that inhibit sCD22. Any suitable antibody (e.g., monoclonal, polyclonal, or synthetic) may be utilized in the therapeutic methods disclosed herein. In some embodiments, the antibodies are humanized antibodies. Methods for humanizing antibodies are well known in the art (See e.g., U.S. Pat. Nos. 6,180,370, 5,585,089, 6,054,297, and 5,565,332; each of which is herein incorporated by reference).

The present invention is not limited to the use of any particular antibody configuration. In some preferred embodiments, the targeting unit is an antigen binding protein. Preferred antigen binding proteins include, but are not limited to an immunoglobulins, a Fab, F(ab′)₂, Fab′ single chain antibody, Fv, single chain (scFv), mono-specific antibody, bi-specific antibody, tri-specific antibody, multivalent antibody, chimeric antibody, humanized antibody, human antibody, CDR-grafted antibody, shark antibody, an immunoglobulin single variable domain (e.g., a nanobody or a single variable domain antibody), minibody, camelid antibody (e.g., from the Camelidae family) microbody, intrabody (e.g., intracellular antibody), and/or de-fucosylated antibody and/or derivative thereof. Mimetics of binding agents and/or antibodies are also provided.

In some embodiments, scFv polypeptides described herein are fused to Fc regions to generate minibodies. As used herein, the term “fragment crystallizable region (Fc region)” refers to the tail region of an antibody that interacts with cell surface receptors called Fc receptors and some proteins of the complement system. This property allows antibodies to activate the immune system. In IgG, IgA and IgD antibody isotypes, the Fc region is composed of two identical protein fragments, derived from the second and third constant domains of the antibody's two heavy chains; IgM and IgE Fc regions contain three heavy chain constant domains (CH domains 2-4) in each polypeptide chain. The Fc regions of IgGs bear a highly conserved N-glycosylation site.

In some embodiments, the Fc region is derived from an IgG. In some embodiments, the IgG is human IgG1, although other suitable Fc regions derived from other organisms or antibody frameworks may be utilized.

In some embodiments, scFv polypeptides described herein are fused to chimeric antigen receptors. Chimeric antigen receptors (CARs), (also known as chimeric immunoreceptors, chimeric T cell receptors, artificial T cell receptors or CAR-T) are engineered receptors, which graft an arbitrary specificity onto an immune effector cell (T cell). Typically, these receptors are used to graft the specificity of an antibody (e.g., an scFv described herein) onto a T cell, with transfer of their coding sequence facilitated by retroviral vectors. The receptors are called chimeric because they are composed of parts from different sources.

Further, the present invention also envisages expression vectors comprising nucleic acid sequences encoding any of the above polypeptides or fusion proteins thereof or functional fragments thereof, as well as host cells expressing such expression vectors. Suitable expression systems include constitutive and inducible expression systems in bacteria or yeasts, virus expression systems, such as baculovirus, semliki forest virus and lentiviruses, or transient transfection in insect or mammalian cells. Suitable host cells include E. coli, Lactococcus lactis, Saccharomyces cerevisiae, Schizosaccharomyces pombe, Pichia pastoris, and the like. Suitable animal host cells include HEK 293, COS, S2, CHO, NSO, DT40 and the like. The cloning, expression and/or purification of the antibodies can be done according to techniques known by the skilled person in the art.

It will be understood that polypeptides described herein may be identified with reference to the nucleotide and/or amino acid sequence corresponding to the variable and/or complementarity determining regions (“CDRs”) thereof.

Also within the scope of the invention are natural or synthetic analogs, mutants, variants, alleles, homologs and orthologs (herein collectively referred to as “variants”) of the immunoglobulin single variable domains of the invention as defined herein. Thus, according to one embodiment of the invention, the term “immunoglobulin single variable domain of the invention” in its broadest sense also covers such variants, in particular variants of the antibodies described herein. Generally, in such variants, one or more amino acid residues may have been replaced, deleted and/or added compared to the antibodies of the invention as defined herein. Such substitutions, insertions or deletions may be made in one or more of the framework regions and/or in one or more of the CDRs. Variants, as used herein, are sequences wherein each or any framework region and each or any complementarity determining region shows at least 80% identity, preferably at least 85% identity, more preferably 90% identity, even more preferably 95% identity or, still even more preferably 99% identity with the corresponding region in the reference sequence (i.e., FR1_variant versus FR1_reference, CDR1_variant versus CDR1_reference, FR2_variant versus FR2_reference, CDR2_variant versus CDR2_reference, FR3_variant versus FR3_reference, CDR3_variant versus CDR3_reference, FR4_variant versus FR4_reference), as can be measured electronically by making use of algorithms such as PILEUP and BLAST. (See, e.g., Higgins & Sharp, CABIOS 5:151 (1989); Altschul S. F., W. Gish, W. Miller, E. W. Myers, D. J. Lipman. Basic local alignment search tool. J. Mol. Biol. 1990; 215:403-10.) Software for performing BLAST analyses is publicly available through the National Center for Biotechnology Information (on the worldwide web at ncbi.nlm.nih.gov/). Such variants of immunoglobulin single variable domains may be of particular advantage since they may have improved potency or other desired properties.

A “deletion” is defined here as a change in either amino acid or nucleotide sequence in which one or more amino acid or nucleotide residues, respectively, are absent as compared to an amino acid sequence or nucleotide sequence of a parental polypeptide or nucleic acid. Within the context of a protein, a deletion can involve deletion of about two, about five, about ten, up to about twenty, up to about thirty or up to about fifty or more amino acids. A protein or a fragment thereof may contain more than one deletion.

An “insertion” or “addition” is that change in an amino acid or nucleotide sequences which has resulted in the addition of one or more amino acid or nucleotide residues, respectively, as compared to an amino acid sequence or nucleotide sequence of a parental protein. “Insertion” generally refers to addition to one or more amino acid residues within an amino acid sequence of a polypeptide, while “addition” can be an insertion or refer to amino acid residues added at an N- or C-terminus, or both termini. Within the context of a protein or a fragment thereof, an insertion or addition is usually of about one, about three, about five, about ten, up to about twenty, up to about thirty or up to about fifty or more amino acids. A protein or fragment thereof may contain more than one insertion.

A “substitution,” as used herein, results from the replacement of one or more amino acids or nucleotides by different amino acids or nucleotides, respectively as compared to an amino acid sequence or nucleotide sequence of a parental protein or a fragment thereof. It is understood that a protein or a fragment thereof may have conservative amino acid substitutions which have substantially no effect on the protein's activity. By conservative substitutions is intended combinations such as gly, ala; val, ile, leu, met; asp, glu; asn, gln; ser, thr; lys, arg; cys, met; and phe, tyr, trp.

By means of non-limiting examples, a substitution may, for example, be a conservative substitution (as described herein) and/or an amino acid residue may be replaced by another amino acid residue that naturally occurs at the same position in another variable domain. Thus, any one or more substitutions, deletions or insertions, or any combination thereof, that either improve the properties of the antibody of the invention or that at least do not detract too much from the desired properties or from the balance or combination of desired properties of the antibody of the invention (i.e., to the extent that the antibody is no longer suited for its intended use) are included within the scope of the invention. A skilled person will generally be able to determine and select suitable substitutions, deletions or insertions, or suitable combinations of thereof, based on the disclosure herein and optionally after a limited degree of routine experimentation, which may, for example, involve introducing a limited number of possible substitutions and determining their influence on the properties of the antibodies thus obtained.

Further, depending on the host organism used to express the immunoglobulin single variable domain of the invention, such deletions and/or substitutions may be designed in such a way that one or more sites for post-translational modification (such as one or more glycosylation sites) are removed, as will be within the ability of the person skilled in the art. Alternatively, substitutions or insertions may be designed so as to introduce one or more sites for attachment of functional groups (as described herein), for example, to allow site-specific pegylation.

Examples of modifications, as well as examples of amino acid residues within the immunoglobulin single variable domain, that can be modified (i.e., either on the protein backbone but preferably on a side chain), methods and techniques that can be used to introduce such modifications and the potential uses and advantages of such modifications will be clear to the skilled person. For example, such a modification may involve the introduction (e.g., by covalent linking or in another suitable manner) of one or more functional groups, residues or moieties into or onto the immunoglobulin single variable domain of the invention, and in particular of one or more functional groups, residues or moieties that confer one or more desired properties or functionalities to the immunoglobulin single variable domain of the invention. Examples of such functional groups and of techniques for introducing them will be clear to the skilled person, and can generally comprise all functional groups and techniques mentioned in the general background art cited hereinabove as well as the functional groups and techniques known per se for the modification of pharmaceutical proteins, and in particular for the modification of antibodies or antibody fragments (including ScFvs and single domain antibodies), for which reference is, for example, made to Remington's Pharmaceutical Sciences, 16th ed., Mack Publishing Co., Easton, Pa. (1980). Such functional groups may, for example, be linked directly (for example, covalently) to an immunoglobulin single variable domain of the invention, or optionally via a suitable linker or spacer, as will again be clear to the skilled person. One of the most widely used techniques for increasing the half-life and/or reducing immunogenicity of pharmaceutical proteins comprises attachment of a suitable pharmacologically acceptable polymer, such as poly(ethyleneglycol) (PEG) or derivatives thereof (such as methoxypoly(ethyleneglycol) or mPEG). Generally, any suitable form of pegylation can be used, such as the pegylation used in the art for antibodies and antibody fragments (including but not limited to (single) domain antibodies and ScFvs); reference is made to, for example, Chapman, Nat. Biotechnol., 54, 531-545 (2002); by Veronese and Harris, Adv. Drug Deliv. Rev. 54, 453-456 (2003), by Harris and Chess, Nat. Rev. Drug. Discov., 2, (2003) and in WO04060965. Various reagents for pegylation of proteins are also commercially available, for example, from Nektar Therapeutics, USA. Preferably, site-directed pegylation is used, in particular via a cysteine-residue (see, for example, Yang et al., Protein Engineering, 16, 10, 761-770 (2003). For example, for this purpose, PEG may be attached to a cysteine residue that naturally occurs in an antibody of the invention, an antibody of the invention may be modified so as to suitably introduce one or more cysteine residues for attachment of PEG, or an amino acid sequence comprising one or more cysteine residues for attachment of PEG may be fused to the N- and/or C-terminus of a antibody of the invention, all using techniques of protein engineering known per se to the skilled person. Preferably, for the immunoglobulin single variable domains and proteins of the invention, a PEG is used with a molecular weight of more than 5000, such as more than 10,000 and less than 200,000, such as less than 100,000; for example, in the range of 20,000-80,000. Another, usually less preferred modification comprises N-linked or O-linked glycosylation, usually as part of co-translational and/or post-translational modification, depending on the host cell used for expressing the immunoglobulin single variable domain or polypeptide of the invention. Another technique for increasing the half-life of an immunoglobulin single variable domain may comprise the engineering into bifunctional constructs or into fusions of immunoglobulin single variable domains with peptides (for example, a peptide against a serum protein such as albumin).

Yet another modification may comprise the introduction of one or more detectable labels or other signal-generating groups or moieties, depending on the intended use of the labeled antibody. Suitable labels and techniques for attaching, using and detecting them will be clear to the skilled person and, for example, include, but are not limited to, fluorescent labels (such as fluorescein, isothiocyanate, rhodamine, phycoerythrin, phycocyanin, allophycocyanin, o-phthaldehyde, and fluorescamine and fluorescent metals such as Eu or others metals from the lanthanide series), phosphorescent labels, chemiluminescent labels or bioluminescent labels (such as luminal, isoluminol, theromatic acridinium ester, imidazole, acridinium salts, oxalate ester, dioxetane or GFP and its analogs), radio-isotopes, metals, metals chelates or metallic cations or other metals or metallic cations that are particularly suited for use in in vivo, in vitro or in situ diagnosis and imaging, as well as chromophores and enzymes (such as malate dehydrogenase, staphylococcal nuclease, delta-V-steroid isomerase, yeast alcohol dehydrogenase, alpha-glycerophosphate dehydrogenase, triose phosphate isomerase, biotinavidin peroxidase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholine esterase). Other suitable labels will be clear to the skilled person and, for example, include moieties that can be detected using NMR or ESR spectroscopy. Such labeled antibodies and polypeptides of the invention may, for example, be used for in vitro, in vivo or in situ assays (including immunoassays known per se such as ELISA, RIA, EIA and other “sandwich assays,” etc.), as well as in vivo diagnostic and imaging purposes, depending on the choice of the specific label. As will be clear to the skilled person, another modification may involve the introduction of a chelating group, for example, to chelate one of the metals or metallic cations referred to above. Suitable chelating groups, for example, include, without limitation, diethyl-enetriaminepentaacetic acid (DTPA) or ethylenediaminetetraacetic acid (EDTA). Yet another modification may comprise the introduction of a functional group that is one part of a specific binding pair, such as the biotin-(strept)avidin binding pair. Such a functional group may be used to link the antibody of the invention to another protein, polypeptide or chemical compound that is bound to the other half of the binding pair, i.e., through formation of the binding pair. For example, an antibody of the invention may be conjugated to biotin, and linked to another protein, polypeptide, compound or carrier conjugated to avidin or streptavidin. For example, such a conjugated antibody may be used as a reporter, for example, in a diagnostic system where a detectable signal-producing agent is conjugated to avidin or streptavidin. Such binding pairs may, for example, also be used to bind the antibody of the invention to a carrier, including carriers suitable for pharmaceutical purposes. One non-limiting example are the liposomal formulations described by Cao and Suresh, Journal of Drug Targeting, 8, 4, 257 (2000). Such binding pairs may also be used to link a therapeutically active agent to the antibody of the invention.

In some embodiments, the immunoglobulin single variable domain of the present invention is fused to a detectable label, either directly or through a linker. Preferably, the detectable label is a radio-isotope or radioactive tracer, which is suitable for medical applications, such as in in vivo nuclear imaging. Examples include, without the purpose of being limitative, ⁹⁹mTc, ¹²³I, ¹²⁵I, ¹¹¹In, ¹⁸F, ⁶⁴Cu, ⁶⁷Ga, ⁶⁸Ga, and any other radio-isotope which can be used in animals, in particular mouse or human.

In still another embodiment, the immunoglobulin single variable domain of the present invention is fused to a moiety selected from the group consisting of a toxin, or to a cytotoxic drug, or to an enzyme capable of converting a prodrug into a cytotoxic drug, or to a radionuclide, or coupled to a cytotoxic cell, either directly or through a linker.

In some embodiments, the present invention provides an antibody-drug conjugate and/or an antibody-enzyme conjugate comprising, for example, a CD22 antibody linked to 3Fax-NeuAc, and/or a CD22 antibody linked to a sialidase. In certain embodiments, the antibody drug conjugates are administered to cells expressing CD22.

As used herein, “linkers” are peptides of 1 to 50 amino acids length and are typically chosen or designed to be unstructured and flexible. These include, but are not limited to, synthetic peptides rich in Gly, Ser, Thr, Gln, Glu or further amino acids that are frequently associated with unstructured regions in natural proteins. (See, e.g., Dosztanyi Z., V. Csizmok, P. Tompa, and I. Simon (2005). IUPred: web server for the prediction of intrinsically unstructured regions of proteins based on estimated energy content. Bioinformatics (Oxford, England), 21(16), 3433-4.)

Monoclonal antibodies against target antigens (e.g., CD22, IGF2R) are produced by a variety of techniques including conventional monoclonal antibody methodologies such as the somatic cell hybridization techniques of Kohler and Milstein, Nature, 256:495 (1975). Although in some embodiments, somatic cell hybridization procedures are preferred, other techniques for producing monoclonal antibodies are contemplated as well (e.g., viral or oncogenic transformation of B lymphocytes).

A preferred animal system for preparing hybridomas is the murine system. Hybridoma production in the mouse is a well-established procedure. Immunization protocols and techniques for isolation of immunized splenocytes for fusion are known in the art. Fusion partners (e.g., murine myeloma cells) and fusion procedures are also known.

Human monoclonal antibodies (mAbs) directed against human proteins can be generated using transgenic mice carrying the complete human immune system rather than-the mouse system. Splenocytes from the transgenic mice are immunized with the antigen of interest, which are used to produce hybridomas that secrete human mAbs with specific affinities for epitopes from a human protein. (See e.g., Wood et al., WO 91/00906, Kucherlapati et al., WO 91/10741; Lonberg et al., WO 92/03918; Kay et al., WO 92/03917 (each of which is herein incorporated by reference in its entirety); N. Lonberg et al., Nature, 368:856-859 [1994]; L. L. Green et al., Nature Genet., 7:13-21 [1994]; S. L. Morrison et al., Proc. Nat. Acad. Sci. USA, 81:6851-6855 [1994]; Bruggeman et al., Immunol., 7:33-40 [1993]; Tuaillon et al., Proc. Nat. Acad. Sci. USA, 90:3720-3724 [1993]; and Bruggernan et al. Eur. J. Immunol., 21:1323-1326 [1991]).

Monoclonal antibodies can also be generated by other methods known to those skilled in the art of recombinant DNA technology. An alternative method, referred to as the “combinatorial antibody display” method, has been developed to identify and isolate antibody fragments having a particular antigen specificity, and can be utilized to produce monoclonal antibodies. (See e.g., Sastry et al., Proc. Nat. Acad. Sci. USA, 86:5728 [1989]; Huse et al., Science, 246:1275 [1989]; and Orlandi et al., Proc. Nat. Acad. Sci. USA, 86:3833 [1989]). After immunizing an animal with an immunogen as described above, the antibody repertoire of the resulting B-cell pool is cloned. Methods are generally known for obtaining the DNA sequence of the variable regions of a diverse population of immunoglobulin molecules by using a mixture of oligomer primers and the PCR. For instance, mixed oligonucleotide primers corresponding to the 5′ leader (signal peptide) sequences and/or framework 1 (FR1) sequences, as well as primer to a conserved 3′ constant region primer can be used for PCR amplification of the heavy and light chain variable regions from a number of murine antibodies. (See e.g., Larrick et al., Biotechniques, 11:152-156 [1991]). A similar strategy can also be used to amplify human heavy and light chain variable regions from human antibodies (See e.g., Larrick et al., Methods: Companion to Methods in Enzymology, 2:106-110 [1991]).

The term modified antibody is also intended to include antibodies, such as monoclonal antibodies, chimeric antibodies, and humanized antibodies which have been modified by, for example, deleting, adding, or substituting portions of the antibody. For example, an antibody can be modified by deleting the hinge region, thus generating a monovalent antibody. Any modification is within the scope of the invention so long as the antibody has at least one antigen binding region specific.

Chimeric mouse-human monoclonal antibodies can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted. (See e.g., Robinson et al., PCT/US86/02269; European Patent Application 184,187; European Patent Application 171,496; European Patent Application 173,494; WO 86/01533; U.S. Pat. No. 4,816,567; European Patent Application 125,023 [each of which is herein incorporated by reference in its entirety]; Better et al., Science, 240:1041-1043 [1988]; Liu et al., Proc. Nat. Acad. Sci. USA, 84:3439-3443 [1987]; Liu et al., J. Immunol., 139:3521-3526 [1987]; Sun et al., Proc. Nat. Acad. Sci. USA, 84:214-218 [1987]; Nishimura et al., Canc. Res., 47:999-1005 [1987]; Wood et al., Nature, 314:446-449 [1985]; and Shaw et al., J. Natl. Cancer Inst., 80:1553-1559 [1988]).

The chimeric antibody can be further humanized by replacing sequences of the Fv variable region that are not directly involved in antigen binding with equivalent sequences from human Fv variable regions. General reviews of humanized chimeric antibodies are provided by S. L. Morrison, Science, 229:1202-1207 (1985) and by Oi et al., Bio. Techniques, 4:214 (1986). Those methods include isolating, manipulating, and expressing the nucleic acid sequences that encode all or part of immunoglobulin Fv variable regions from at least one of a heavy or light chain.

Suitable humanized antibodies can alternatively be produced by CDR substitution (e.g., U.S. Pat. No. 5,225,539 (incorporated herein by reference in its entirety); Jones et al., Nature, 321:552-525 [1986]; Verhoeyan et al., Science, 239:1534 [1988]; and Beidler et al., J. Immunol., 141:4053 [1988]). All of the CDRs of a particular human antibody may be replaced with at least a portion of a non-human CDR or only some of the CDRs may be replaced with non-human CDRs. It is only necessary to replace the number of CDRs required for binding of the humanized antibody to the Fc receptor.

An antibody can be humanized by any method that is capable of replacing at least a portion of a CDR of a human antibody with a CDR derived from a non-human antibody. The human CDRs may be replaced with non-human CDRs; using oligonucleotide site-directed mutagenesis.

Also within the scope of the invention are chimeric and humanized antibodies in which specific amino acids have been substituted, deleted or added. In particular, preferred humanized antibodies have amino acid substitutions in the framework region, such as to improve binding to the antigen. For example, in a humanized antibody having mouse CDRs, amino acids located in the human framework region can be replaced with the amino acids located at the corresponding positions in the mouse antibody. Such substitutions are known to improve binding of humanized antibodies to the antigen in some instances.

The antibodies can be of various isotypes, including, but not limited to: IgG (e.g., IgG1, IgG2, IgG2a, IgG2b, IgG2c, IgG3, IgG4); IgM; IgA1; IgA2; IgA_{sec}; IgD; and IgE. In some preferred embodiments, the antibody is an IgG isotype. In other preferred embodiments, the antibody is an IgM isotype. The antibodies can be full-length (e.g., an IgG1, IgG2, IgG3, or IgG4 antibody) or can include only an antigen-binding portion (e.g., a Fab, F(ab′)₂, Fv or a single chain Fv fragment).

In preferred embodiments, the immunoglobulin is a recombinant antibody (e.g., a chimeric or a humanized antibody), a subunit, or an antigen binding fragment thereof (e.g., has a variable region, or at least a complementarity determining region (CDR)).

In some embodiments, the immunoglobulin is monovalent (e.g., includes one pair of heavy and light chains, or antigen binding portions thereof). In other embodiments, the immunoglobulin is a divalent (e.g., includes two pairs of heavy and light chains, or antigen binding portions thereof).

### Oligomeric Antisense Compounds and Allele Specific Oligonucleotides

In some embodiments, compositions comprising oligomeric antisense compounds, particularly oligonucleotides are used to modulate the function of nucleic acid molecules encoding CD22, thereby modulating the amount of CD22 expressed. This is accomplished by providing antisense compounds that specifically hybridize with one or more nucleic acids encoding CD22. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of CD22. In the context of the present disclosure, “modulation” means either an increase (stimulation) or a decrease (inhibition) in the expression of a gene. For example, CD22 expression may be inhibited to treat or prevent a dementia, atherosclerosis or cancer.

In some embodiments, nucleic acids are siRNAs. “RNA interference (RNAi)” is the process of sequence-specific, post-transcriptional gene silencing initiated by a small interfering RNA (siRNA). During RNAi, siRNA induces degradation of target mRNA with consequent sequence-specific inhibition of gene expression.

An “RNA interference,” “RNAi,” “small interfering RNA” or “short interfering RNA” or “siRNA” or “short hairpin RNA” or “shRNA” molecule, or “miRNA” is a RNA duplex of nucleotides that is targeted to a nucleic acid sequence of interest, for example, CD22. As used herein, the term “siRNA” is a generic term that encompasses all possible RNAi triggers. An “RNA duplex” refers to the structure formed by the complementary pairing between two regions of a RNA molecule. siRNA is “targeted” to a gene in that the nucleotide sequence of the duplex portion of the siRNA is complementary to a nucleotide sequence of the targeted gene. certain embodiments, the siRNAs are targeted to the sequence encoding CD22. In some embodiments, the length of the duplex of siRNAs is less than 30 base pairs. In some embodiments, the duplex can be 32, 31, 30, 29, 28, 27, 26, 25, 24, 23, 22, 21, 20, 19, 18, 17, 16, 15, 14, 13, 12, 11 or 10 base pairs in length. In some embodiments, the length of the duplex is 19 to 32 base pairs in length. In certain embodiment, the length of the duplex is 19 or 21 base pairs in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12, 13, 14, 15, 16, 17, 18, 19, 20, 21, 22, 23, 24, 25, 26 or 27 nucleotides in length. In certain embodiments, the loop is 18 nucleotides in length. The hairpin structure can also contain 3′ and/or 5′ overhang portions. In some embodiments, the overhang is a 3′ and/or a 5′ overhang 0, 1, 2, 3, 4 or 5 nucleotides in length.

As used herein, Dicer-substrate RNAs (DsiRNAs) are chemically synthesized asymmetric 25-mer/27-mer duplex RNAs that have increased potency in RNA interference compared to traditional siRNAs. Traditional 21-mer siRNAs are designed to mimic Dicer products and therefore bypass interaction with the enzyme Dicer. Dicer has been recently shown to be a component of RISC and involved with entry of the siRNA duplex into RISC. Dicer-substrate siRNAs are designed to be optimally processed by Dicer and show increased potency by engaging this natural processing pathway. Using this approach, sustained knockdown has been regularly achieved using sub-nanomolar concentrations. (U.S. Pat. No. 8,084,599; Kim et al., Nature Biotechnology 23 :222 2005; Rose et al., Nucleic Acids Res., 33:4140 2005).

The transcriptional unit of a “shRNA” is comprised of sense and antisense sequences connected by a loop of unpaired nucleotides. shRNAs are exported from the nucleus by Exportin-5, and once in the cytoplasm, are processed by Dicer to generate functional siRNAs. “miRNAs” stem-loops are comprised of sense and antisense sequences connected by a loop of unpaired nucleotides typically expressed as part of larger primary transcripts (pri-miRNAs), which are excised by the Drosha-DGCR8 complex generating intermediates known as pre-miRNAs, which are subsequently exported from the nucleus by Exportin-5, and once in the cytoplasm, are processed by Dicer to generate functional miRNAs or siRNAs. “Artificial miRNA” or an “artificial miRNA shuttle vector”, as used herein interchangeably, refers to a primary miRNA transcript that has had a region of the duplex stem loop (at least about 9-20 nucleotides) which is excised via Drosha and Dicer processing replaced with the siRNA sequences for the target gene while retaining the structural elements within the stem loop necessary for effective Drosha processing. The term “artificial” arises from the fact the flanking sequences (about 35 nucleotides upstream and .about.40 nucleotides downstream) arise from restriction enzyme sites within the multiple cloning site of the siRNA. As used herein the term “miRNA” encompasses both the naturally occurring miRNA sequences as well as artificially generated miRNA shuttle vectors.

The siRNA can be encoded by a nucleic acid sequence, and the nucleic acid sequence can also include a promoter. The nucleic acid sequence can also include a polyadenylation signal. In some embodiments, the polyadenylation signal is a synthetic minimal polyadenylation signal or a sequence of six Ts.

The present disclosure contemplates the use of any genetic manipulation for use in modulating the expression of CD22. Examples of genetic manipulation include, but are not limited to, gene knockout (e.g., removing the CD22 gene from the chromosome using, for example, recombination), expression of antisense constructs with or without inducible promoters, and the like. Delivery of nucleic acid construct to cells in vitro or in vivo may be conducted using any suitable method. A suitable method is one that introduces the nucleic acid construct into the cell such that the desired event occurs (e.g., expression of an antisense construct).

Introduction of molecules carrying genetic information into cells is achieved by any of various methods including, but not limited to, directed injection of naked DNA constructs, bombardment with gold particles loaded with said constructs, and macromolecule mediated gene transfer using, for example, liposomes, biopolymers, and the like. Exemplary methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer into a variety of solid tumors in animal models and into human solid tumor xenografts in immune-deficient mice. Examples of adenoviral vectors and methods for gene transfer are described in PCT publications WO 00/12738 and WO 00/09675 and U.S. Pat. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety.

Vectors may be administered to subject in a variety of ways. For example, in some embodiments of the present disclosure, vectors are administered into tumors or tissue associated with tumors using direct injection. In other embodiments, administration is via the blood or lymphatic circulation (See e.g., PCT publication 1999/02685 herein incorporated by reference in its entirety). Exemplary dose levels of adenoviral vector are preferably 10⁸ to 10¹¹ vector particles added to the perfusate.

### Peptide Targeted Therapeutics and Fusion Proteins

In some embodiments, methods and compositions of the present invention comprise de novo peptide targeted therapeutics as described, for example, by Chevalier A. et al. Nature Publishing Group 2017:550;74-79 incorporated by reference herein in its entirety.

In some embodiments, recombinant CD22 fusion proteins that block sialosides are provided. Where clinical applications are contemplated, in some embodiments of the present invention, the fusion proteins are prepared as part of a pharmaceutical composition in a form appropriate for the intended application. Generally, this entails preparing compositions that are essentially free of pyrogens, as well as other impurities that could be harmful to humans or animals. However, in some embodiments of the present invention, a fusion protein composition formulation may be administered using one or more of the routes described herein.

In some embodiments, the fusion protein compositions are used in conjunction with appropriate salts and buffers to render delivery of the compositions in a stable manner to allow for uptake by target cells. Buffers also are employed when the compositions are introduced into a patient. Aqueous compositions comprise an effective amount of composition dispersed in a pharmaceutically acceptable carrier or aqueous medium. Such compositions also are referred to as inocula. In some embodiments, candidate CD22 inhibitors are screened for activity (e.g., using the methods described in the experimental methods or another suitable assay). In certain embodiments, the CD22 inhibitor is a sialoside. In particular embodiments, the sialoside is a high affinity synthetic sialic acid analog and/or selective Siglec glycan ligand including, for example, A 2,3-Dichlorobenzyl derivative, a o-nosyl (ortho-nitrophenylsulfonyl) derivative, a sialoside that contains a dichlorobenzyl substituent at the anomeric position, an ortho-nitrobenzylsulfonamide at the 5-position, and a 4′-hydroxy-4-biphenylcarboxamide at the 9-position, a α-9-N-(biphenyl-4-carbonyl)-amino-9-deoxy-Neu5Ac (BPC-Neu5Ac), a 9-BPC-Neu5Acα2-6Galβ1-4GlcNAc (BPC-Neu5Ac-LacNAc), or a benzyl-Neu5Acα (See, e.g., Mesch et al. ChemMedChem 7:134-143, 2012, Angata et al. Trends Pharmacological Science 36:645-660, 2015, and Bull et al. Trends in Biochemical Sciences 41:519-531, 2016.) In particular embodiments, cell permeation is enhanced by administration of a high affinity and selectivity glycan ligand as a prodrug, by replacing, for example, the carboxylate with a bioisostere, or by administering high affinity CD22 ligands on the surface of liposomal nanoparticles. Additional CD22 in inhibitors comprise those described by Kelm, S. et al. J Exp Med 2002: 195, 1207-1213, Duong, B. H. et al. J Exp Med 2010:207, 173-187, Collins, B. E. et al. The Journal of Immunology 2006:177, 2994-3003, Kelm, S. et al. Angew. Chem. Int. Ed. 2013:52, 3616-3620, Rillahan, C. D. et al. Chem. Sci. 2014:5, 2398-17, and Mesch, S. et al. ChemMedChem 2011: 7, 134-143, each of which is incorporated herein in its entirety.

### CNS Delivery

In some embodiments, the CD22 inhibitors and agents are delivered to the CNS by methods and compositions that promote transfer across the blood brain barrier (BBB). In certain embodiments, the methods and compositions comprise one or more bi-specific antibodies comprising, for example, antibodies to highly expressed proteins, including basigin, Glut1, and CD98hc. Antibodies to these targets are significantly enriched in the brain after administration in vivo. In particular, antibodies against CD98hc show robust accumulation in brain after systemic dosing. Accordingly, in specific embodiments, methods and compositions of the present invention comprise, for example, use of CD98hc as a robust receptor-mediated transcytosis pathway for antibody delivery to the brain. (Zuchero et al. Neuron 89;70-82, 2016.) In further embodiments, transfer across the BBB is enhanced by transient disruption, for example, osmotic or pharmacologic disruption, and/or by other membrane protein pathways using receptor-mediate transcytosis comprising, for example, antibodies against the transferrin receptor.

### Pharmaceutical Compositions and Formulations

The present disclosure further provides pharmaceutical compositions (e.g., comprising the compounds described above). The pharmaceutical compositions of the present disclosure may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intra-arterial, subcutaneous, intraperitoneal or intramuscular injection or infusion; or intracranial, e.g., intrathecal or intraventricular, administration. In certain embodiments, CD22 inhibitors and agents that decrease cell surface sialic acid are administered by methods that bypass the BBB including, for example, direct application to the surface of the CNS, to the parenchyma of the CNS, to the ventricles of the CNS, and to the cerebrospinal fluid (CSF) of the CNS. In particular, intrathecal and epidural administration may be achieved by single shot, a series of single shots, and/or by continuous administration to the CSF. In certain embodiments, continuous administration to the CSF is provided by a programmable external pump. In other embodiments, continuous administration is provided by a programmable implantable pump.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present disclosure include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present disclosure, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present disclosure may be formulated into any of many possible dosage forms such as, but not limited to, tablets, capsules, liquid syrups, soft gels, suppositories, and enemas. The compositions of the present disclosure may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

Agents that enhance uptake of oligonucleotides at the cellular level may also be added to the pharmaceutical and other compositions of the present disclosure. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present disclosure may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present disclosure, such as dyes, flavoring agents, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC50s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligonucleotide is administered in maintenance doses, ranging from 0.01 μg to 100 g per kg of body weight, once or more daily, to once every 20 years.

### EXPERIMENTAL EXAMPLES

The following examples are provided in order to demonstrate and further illustrate certain embodiments and aspects of the present disclosure and are not to be construed as limiting the scope thereof.

Oligodendrocyte-Derived sCD22 Binds to Sialic Acid-Dependent Ligands on Microglia

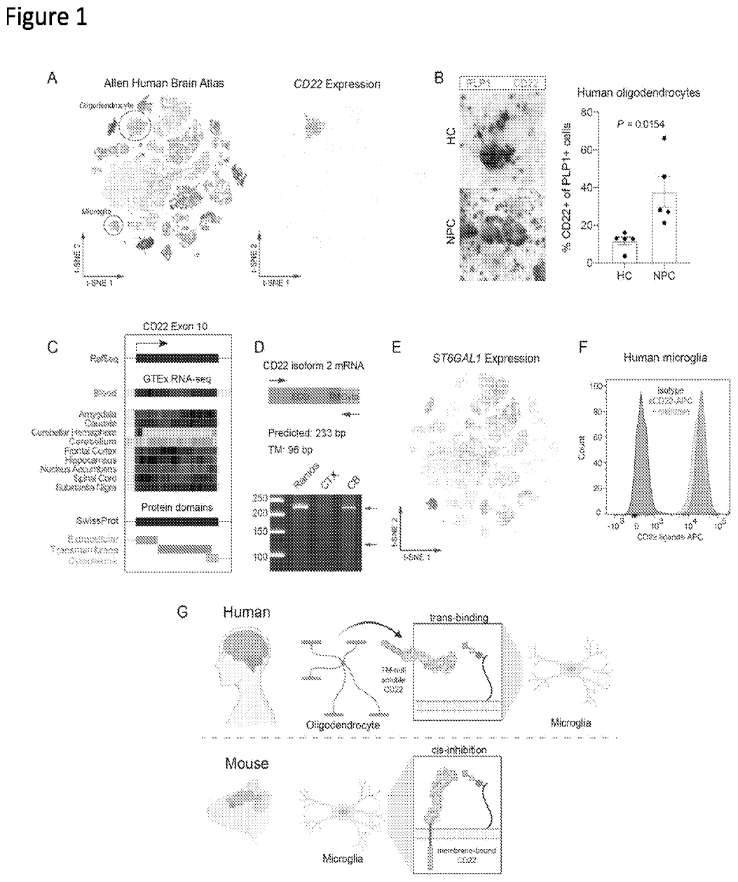

In experiments conducted in the course of development of certain embodiments of the present invention, human snRNA-seq atlases were mined to determine the source of sCD22 in the CSF of patients with NPC¹². In the human brain, CD22 is almost exclusively expressed by oligodendrocytes (FIG. 1a) in contrast to CD22 expression by microglia in the mouse brain, reinforcing divergence of microglial phenotypes between species. To confirm CD22 expression in oligodendrocytes, multiplexed RNA in situ hybridization (RNAscope) on NPC patient and age-matched control cerebella were probed for CD22 and PLP1, a mature oligodendrocyte-specific marker. CD22+ oligodendrocytes were significantly more abundant in the NPC cerebellum (FIG. 1b), and CD22+ puncta were not observed outside PLP1+ oligodendrocytes. These data confirm that CD22 is upregulated in the NPC patient brain in the absence of peripheral immune cell invasion⁷, indicating that oligodendrocytes are the primary source of sCD22 in NPC CSF.

To confirm oligodendrocyte release sCD22 into the CSF, a cerebellum-specific CD22 isoform lacking its transmembrane domain was identified in RNA-seq data from the GTEx. (FIG. 1c). Expression and upregulation of this isoform in the cerebellum, but not cortex, of NPC patients compared to age-matched controls was confirmed by reverse transcription polymerase chain reaction (FIG. 1d).

sCD22 is reported as a biomarker for sepsis and B-cell malignancies^{13,14} and as a biomarker of neurodegeneration in NPC⁷. To determine whether sCD22 binds ligands in the human CNS to modulate brain function, sNuc-seq atlases were mined for expression of ST6GAL1, a glycosyltransferase that attaches sialic acid to growing glycan chains in an α2,6-configuration, the preferred glycan ligand for CD22^{15,16}. ST6GAL1 was observed to be specifically enriched in microglia in the human CNS (FIG. 1e), indicating that ligands on microglia are candidate targets of sCD22. Accordingly, expression of CD22 ligands on fresh human primary microglia was analyzed by flow cytometry using fluorophore-conjugated recombinant CD22 as a staining reagent. Human microglia were observed to express high levels of sCD22 ligands sensitive to sialidase treatment (FIG. 1f), thereby indicating that oligodendrocyte-derived sCD22 binds sialic-acid dependent ligands on microglia in the human CNS (FIG. 1g) compatible with sCD22 modulation of microglial function.

Genetic and Proteomic Screens Identify IGF2R as a sCD22 Ligand on Microglia

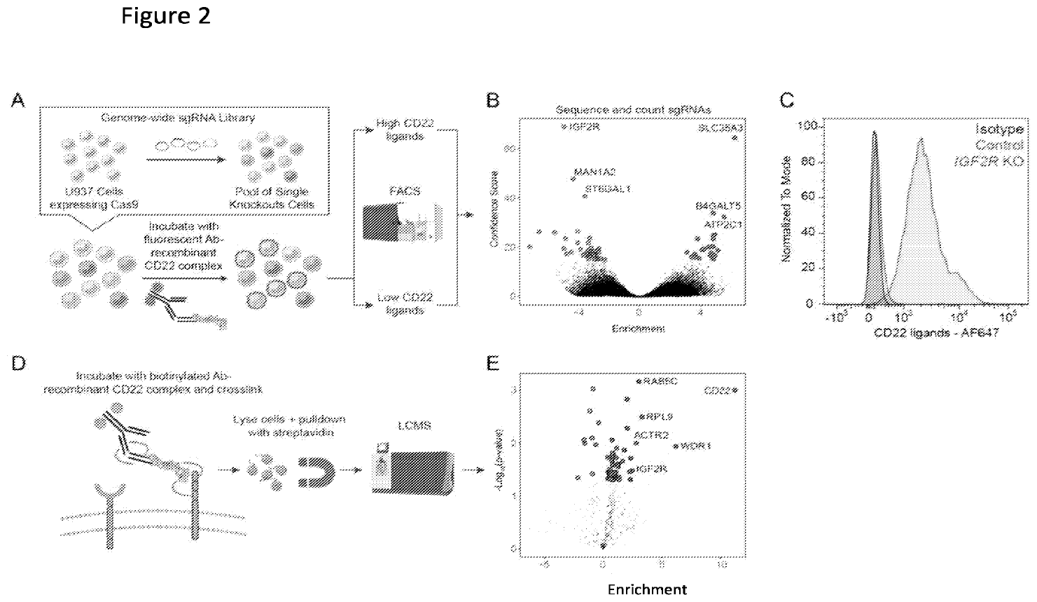

In experiments conducted in the course of development of certain embodiments of the present invention, a genome-wide CRISPR-Cas9 knockout screen was performed using the human myeloid cell line U937 (FIG. 2a) to identify sCD22 ligands on microglia. U937 cells model human microglia¹⁷, and express CD22 ligands. Cas9-expressing U937 cells were infected with a library of single-guide RNAs (sgRNAs) targeting all protein-coding genes, with 10 distinct sgRNAs per gene and ˜10,000 negative control sgRNAs. The pool of stable single-knockout cells was stained with fluorophore-conjugated recombinant CD22, and cells were sorted with high (top 5%) and low (bottom 5%) CD22 ligand expression by fluorescence-activated cell sorting (FACS). Genomic DNA from each population was then sequenced, sgRNA distribution compared between populations, and the effect size and P value for each gene knockout was estimated using casTLE. The screen identified 66 hits with a false discovery rate less than 10%, including ST6GAL1, a known genetic modifier of CD22 glycan ligand synthesis (FIG. 2b). The top hit from the screen was insulin-like growth factor 2 receptor (IGF2R), also referred to as the cation-independent mannose-6-phosphate receptor. IGF2R binds diverse ligands including the growth factor IGF2 and the lysosome-targeting glycan modification mannose-6-phosphate. IGF2R is expressed in primary human microglia. Knocking out IGF2R ablates sCD22 binding to U937 cells (FIG. 2c).

To determine whether IGF2R physically interacts with sCD22, or if it serves an upstream role in displaying the true sCD22 ligand on the cell surface, affinity-purification mass-spectrometry was performed to detect direct binding partners of sCD22 (FIG. 2d). U937 cells were stained with biotinylated recombinant CD22 and treated with a membrane-impermeable cross-linker to stabilize low affinity Siglec-glycan interactions missed by traditional co-immunoprecipitation techniques. Next, cells were lysed, and sCD22-bound protein complexes were enriched with streptavidin beads, stringently washed, and digested for label-free liquid chromatography mass spectrometry (LCMS) analysis. Proteins enriched in the sCD22-bound fraction included IGF2R (FIG. 2e). Taken together, orthogonal genetic and proteomic screens identify IGF2R as a ligand of sCD22 on human microglia.

### CD22 Blocks IGF2R at M6P Binding Sites and Inhibits Lysosomal Trafficking

IGF2R typically binds M6P-tagged proteins in the trans-Golgi network, and releases them in the late endosome. However, a fraction of M6P-tagged proteins are secreted into the extracellular space and recaptured by IGF2R on the cell surface^{18,19}. This minor secretion-recapture pathway is exploited by enzyme-replacement therapy (ERT) to deliver M6P-tagged recombinant proteins to deficient cells²⁰.

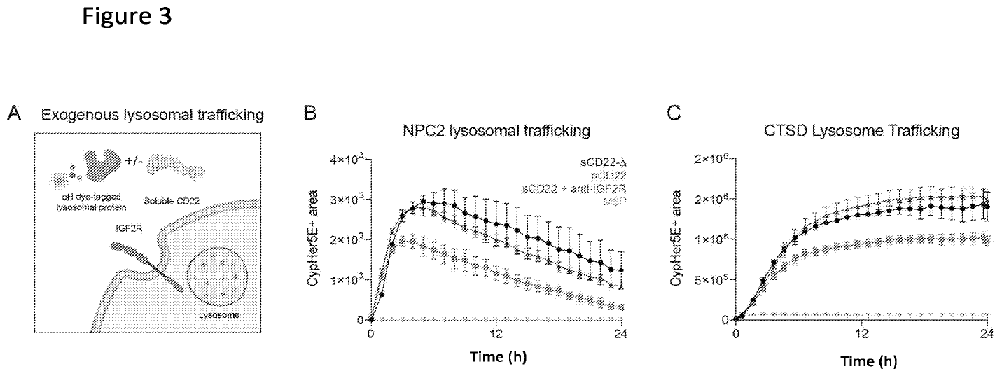

In experiments conducted in the course of development of certain embodiments of the present invention, it was tested whether sCD22 interferes with this pathway through steric hindrance of M6P-binding sites on IGF2R. To assess exogenous lysosomal protein trafficking, a pH-sensitive dye was conjugated to NPC2, the M6P-tagged protein that assists in cholesterol efflux from the lysosome, and that is infrequently mutated in NPC (FIG. 3a). Using IGF2R KO cells or pre-treating WT cells with saturating amounts of free M6P blocked NPC2 trafficking to the lysosome, indicating that IGF2R is the dominant M6PR on U937 cells, and that NPC2 is exclusively trafficked via the M6P pathway. Compared to mutant sCD22 lacking its sialic acid binding domain (sCD22-delta), full-length sCD22 significantly impaired trafficking of NPC2 to the lysosome (FIG. 3b). However, cotreatment with sCD22 and an IGF2R blocking antibody abrogated this effect. Similar results were observed with cathepsin D (FIG. 3c), a lysosomal protease frequently mis-localized in NPC tissues²¹, increased in NPC patient serum²², and shown to have neurotoxic effects^{23,24,25}. Although the invention is not confined to a specific mechanism, results of the experimental examples herein suggest that sCD22 impairs exogenous lysosomal protein trafficking via IGF2R, and exacerbates lysosome protein mis-localization in NPC.

Anti-CD22 Antibodies Disrupt sCD22-IGF2R Interactions and Rescue Lysosomal Dysfunction

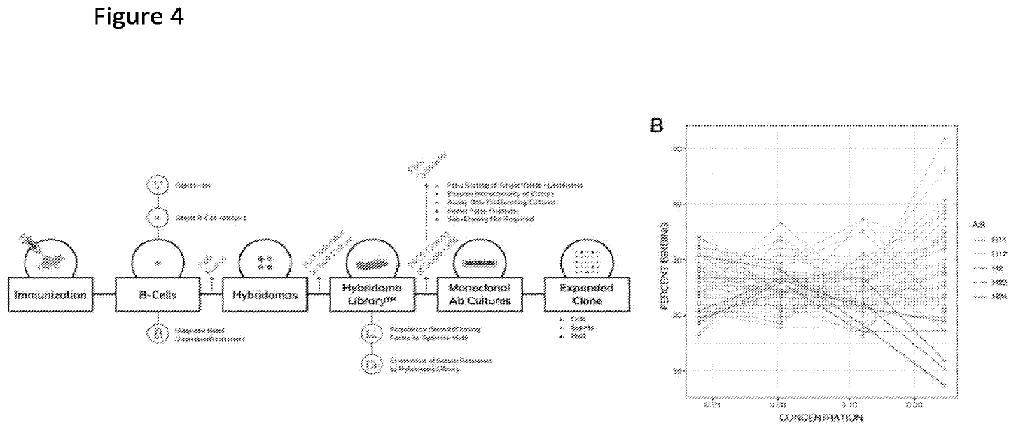

In experiments conducted in the course of development of certain embodiments of the present invention, the capacity of blocking the sCD22 and IGF2R interaction to rescue lysosome dysfunction in NPC tissues was tested. To generate blocking mAbs optimized for targeting sCD22 in the brain, the full-length extracellular domain (ECD) of human CD22 was used as an antigen. Mice were immunized, splenocytes collected, and hybridoma libraries were generated (FIG. 4a). Strong and specific binding of hybridoma supernatants to human CD22 by flow cytometry was confirmed. Hundreds of single hybridoma cells were sorted, and 51 clones selected with intermediate to strong binding. sCD22 was preincubated with each clonal supernatant and exposed the mixture to U937 cells to identify mAbs that potently disrupt sCD22 binding to IGF2R (FIG. 4b).

### REFERENCES

1. de Duve, C., Pressman, B. C., Gianetto, R., Wattiaux, R. & Appelmans, F. Tissue fractionation studies. Intracellular distribution patterns of enzymes in rat-liver tissue. Biochem. J 60, 604-617 (1995).

2. Aharon-Peretz, J., Rosenbaum, H. & Gershoni-Baruch, R. Mutations in the glucocerebrosidase gene and Parkinson's disease in Ashkenazi Jews. N. Engl. J. Med. 351, 1972-1977 (2004)

3. Reddy, J. V., Ganley, I. G. & Pfeffer, S. R. Clues to neuro-degeneration in Niemann-Pick type C disease from global gene expression profiling. PLoS One 1, e19 (2006)

4. Cologna, S. M. et al. Quantitative proteomic analysis of Niemann-Pick disease, type C1 cerebellum identifies protein biomarkers and provides pathological insight. PLoS One 7, e47845 (2012)

5. Walterfang, M. et al. White and gray matter alterations in adults with Niemann-Pick disease type C: a cross-sectional study. Neurology 75, 49-56 (2010)

6. Bianconi, S. E. et al. Evaluation of age of death in Niemann-Pick disease, type C: Utility of disease support group websites to understand natural history. Mol. Genet. Metab. 126, 466-469 (2019)

7. Cougnoux, A. et al. Microglia activation in Niemann—Pick disease, type C1 is amendable to therapeutic intervention. Hum. Mol. Genet. 27, 2076-2089 (2018)

8. Mathys, H. et al. Single-cell transcriptomic analysis of Alzheimer's disease. Nature 570, 332-337 (2019)

9. Zhou, Y. et al. Human and mouse single-nucleus transcriptomics reveal TREM2-dependent and TREM2-independent cellular responses in Alzheimer's disease. Nat. Med. 26, 131-142 (2020)

10. Gerrits, E., Heng, Y., Boddeke, E. W. G. M. & Eggen, B. J. L. Transcriptional profiling of microglia; current state of the art and future perspectives. Glia 68, 740-755 (2020)

11. Pluvinage, J. V. et al. CD22 blockade restores homeostatic microglial phagocytosis in ageing brains. Nature 568, 187-192 (2019)

12. Hodge, R. D. et al. Conserved cell types with divergent features in human versus mouse cortex. Nature (2019) doi:10.1038/s41586-019-1506-7

13. Jiang, Y.-N. et al. Diagnostic and prognostic roles of soluble CD22 in patients with Gram-negative bacterial sepsis. Hepatobiliary Pancreat. Dis. Int 14, 523-529 (2015)

14. Matsushita, K. et al. Soluble CD22 as a tumor marker for hairy cell leukemia. Blood 112, 2272-2277 (2008)

15. Powell, L. D., Sgroi, D., Sjoberg, E. R., Stamenkovic, I. & Varki, A. Natural ligands of the B cell adhesion molecule CD22 beta carry N-linked oligosaccharides with alpha-2,6-linked sialic acids that are required for recognition. J. Biol. Chem. 268, 7019-7027 (1993)

16. Ereno-Orbea, J. et al. Molecular basis of human CD22 function and therapeutic targeting. Nat. Commun. 8, 764 (2017)

17. Haney, M. S. et al. Identification of phagocytosis regulators using magnetic genome-wide CRISPR screens. Nat. Genet. 1-16 (2018)

18. Fratantoni, J. C., Hall, C. W. & Neufeld, E. F. Hurler and Hunter syndromes: mutual correction of the defect in cultured fibroblasts. Science 162, 570-572 (1968)

19. Vladutiu, G. D. & Rattazzi, M. C. Excretion-reuptake route of beta-hexosaminidase in normal and I-cell disease cultured fibroblasts. J. Clin. Invest. 63, 595-601 (1979)

20. Platt, F. M., d'Azzo, A., Davidson, B. L., Neufeld, E. F. & Tifft, C. J. Lysosomal storage diseases. Nat Rev Dis Primers 4, 27 (2018)

21. Amritraj, A. et al. Increased activity and altered subcellular distribution of lysosomal enzymes determine neuronal vulnerability in Niemann-Pick type C1-deficient mice. Am. J. Pathol. 175, 2540-2556 (2009)

22. Cluzeau, C. V. M. et al. Microarray expression analysis and identification of serum biomarkers for Niemann-Pick disease, type C1. Hum. Mol. Genet. 21, 3632-3646 (2012)

23. Kim, S. et al. Neurotoxicity of microglial cathepsin D revealed by secretome analysis. J. Neurochem. 103, 2640-2650 (2007)

24. Cataldo, A. M. & Nixon, R. A. Enzymatically active lysosomal proteases are associated with amyloid deposits in Alzheimer brain. Proc. Natl. Acad. Sci. U.S.A. 87, 3861-3865 (1990)

25. Amritraj, A. et al. Role of cathepsin D in U18666A-induced neuronal cell death: potential implication in Niemann-Pick type C disease pathogenesis. J. Biol. Chem. 288, 3136-3152 (2013)

All publications and patents mentioned in the above specification are herein incorporated by reference. Various modifications and variations of the described method and system of the disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled relevant fields are intended to be within the scope of the following claims.

1. A method of treating a lysosomal storage disorder, comprising: exposing a subject's microglia to a soluble CD22 (sCD22) inhibitor wherein said exposing treats said lysosomal storage disorder.

2. The method of claim 1, wherein said subject is a human subject.

3. The method of claim 1, wherein said lysosomal storage disorder is Niemann-Dick Disease Type C (NPC).

4. The method of claim 1, wherein said sCD22 inhibitor is an anti-sCD22 antibody.

5. The method of claim 1, wherein said sCD22 inhibitor is a sCD22 antagonist.

6. The method of claim 5, wherein said sCD22 antagonist is a glycomimetic compound.

7. The method of claim 5, wherein said sCD22 antagonist is selected from the group consisting of a small molecule, a peptide, and a nucleic acid.

8. The method of claim 1, wherein said sCD22 inhibitor interferes with sCD22 expression.

9. The method of claim 1, wherein said exposing to said sCD22 inhibitor is selected from the group consisting of local administration, topical administration, intrathecal administration, intraparenchymal administration, intracerebroventrical administration, intravenous administration, intraarterial administration, intrapulmonary administration and oral administration.

10. The method of claim 1, wherein said exposing comprises combination therapy with an agent that interferes with NPC1 and/or NPC2 expression.

11. A method of treating a lysosomal storage disorder, comprising: exposing a subject's microglia to an insulin-like growth factor 2 receptor (IGF2R) inhibitor wherein said exposing treats said lysosomal storage disorder.

12. The method of claim 11, wherein said lysosomal storage disorder is Niemann-Pick Disease Type C (NPC).

13. The method of claim 11, wherein said IGF2R inhibitor is an anti-IGFR2 antibody.

14. The method of claim 11, wherein said IGF2R inhibitor is an IGFR2 antagonist.

15. The method of claim 14, wherein said IGF2R antagonist is a glycomimetic compound.

16. The method of claim 14, wherein said IGF2R antagonist is selected from the group consisting of a small molecule, a peptide, and a nucleic acid.

17. The method of claim 11, wherein said IGF2R inhibitor interferes with IGFR2 expression.

18. The method of claim 11, wherein said exposing to said IGF2R inhibitor is selected from the group consisting of local administration, topical administration, intrathecal administration, intraparenchymal administration, intracerebroventrical administration, intravenous administration, intraarterial administration, intrapulmonary administration and oral administration.

19. The method of claim 11, wherein said exposing comprises combination therapy with an agent that interferes with NPC1 and/or NPC2 expression.

20. A composition comprising:
a) a sCD22 antagonist; and
b) an IGF2R antagonist.
